(19) United States
(12) Patent Application Publication
(10) Pub. No.: US 2025/0342918 A1
(43) Pub. Date: Nov. 6, 2025

(54) A SYSTEM FOR IDENTIFYING HYDROGEN STORAGE PROPERTIES OF METAL ALLOYS AND A METHOD THEREOF

(71) Applicant: COUNCIL OF SCIENTIFIC & INDUSTRIAL RESEARCH, New Delhi (IN)

(72) Inventors: Kavita Purushottam JOSHI, Pune (IN); Ashwini Dinanath VERMA, Jammu (IN)

(73) Assignee: COUNCIL OF SCIENTIFIC & INDUSTRIAL RESEARCH, New Delhi (IN)

(21) Appl. No.: 18/866,419

(22) PCT Filed: May 15, 2023
(86) PCT No.: PCT/IN2023/050455

§ 371 Date: Nov. 15, 2024

(30) Foreign Application Priority Data
May 17, 2022 (IN) 202211028454

(51) Int. Cl. G16C 60/00; G16C 20/70

(52) U.S. Cl. CPC G16C 60/00; G16C 20/70

(57) ABSTRACT

The present invention provides an automated method (100) and system (200) for identifying hydrogen storage properties of metal alloys. More particularly, the invention provides a method and system for identification of materials for solid hydrogen storage in multi-component metal alloys. The system (200) can predict hydrogen weight capacity and equilibrium plateau pressure at different temperatures along with enthalpy of hydride formation of multi-component metal alloys with high predictability and case of interpretation. Further, a suitable alloy can be identified by the method (100) employed using the system (200) for hydrogen storage applications based on the hydrogen weight capacity and the equilibrium plateau pressure at different temperatures and enthalpy of hydrogenation, wherein an absorption temperature of the suitable alloy plays a vital role.

### TECHNICAL FIELD OF THE INVENTION

The present disclosure relates to the field of hydrogen storage. More particularly, the present disclosure provides a method and system for identification of materials for solid-state hydrogen storage in multi-component metal alloys as a function of temperature. In particular, the present disclosure provides a system and a method for identifying hydrogen storage properties of metal alloys.

### BACKGROUND OF THE INVENTION

Due to depleting sources of energy, hydrogen has emerged as a major and alternative source of energy in the last couple of decades. Hydrogen is available in abundance in the form of water, biomass, and natural gas. Hydrogen has the highest density of energy per unit weight of any chemical fuel (142 MJKg−1). Furthermore, hydrogen can serve as fuel in many applications like fuel cell vehicles, stationary power generation, thermal systems, and to meet industrial energy need; if stored safely and efficiently.

Several methods are currently employed for hydrogen storage. For physical storage, compressed hydrogen and liquefied hydrogen are the two most common methods. In compressed hydrogen storage, it is compressed in gaseous state under high pressure in a tank. For storing in the liquefied state, the hydrogen must be cooled at subzero temperatures because of its low boiling point and then can be maintained in pressurized and insulated containers. While compressed and liquefied hydrogen are widely utilized in industries, the operational conditions such as high hydrogen pressure and cryogenic temperature often restrict its usage at a wider scale.

The hydrogen can also be stored in selected materials, which is considerably economical and safer than the physical storage techniques. Storing hydrogen in solid-state compounds via chemical absorption results in higher volumetric energy densities than compressed gas or liquid hydrogen. As a result, more hydrogen can be stored in smaller containers, which may be advantageous for portable energy generation. Hydrogen can be stored in materials such as metal hydrides, complex hydrides, high entropy alloys (HEA), etc.

The storage of hydrogen in metal/alloy is a multi-step process that involves the adsorption of molecular hydrogen, followed by dissociation, penetration, and diffusion through the lattice to form the hydride under specific temperature/pressure. Each stage of the process has an energy barrier that influences the hydrogen storage properties. As far as storage in metal alloys is concerned, ideally it requires high hydrogen storage capacity, fast kinetics, and favorable thermodynamics at ambient conditions.

The composition of metal alloys influences efficiency of storing and releasing hydrogen. It has been demonstrated through various studies that the hydrogen storage properties can be modified by altering the composition and structure of hydrides, nano-scaling, and catalyzing the reactions by doping different additives. Hence, continuous attempts have been made to find acceptable as well as best suited materials for solid state hydrogen storage.

Laboratory based efforts to find out the acceptable and best suited solid state materials have certain limitations. The available materials in the chemical registry are infinitely large and, hence, trial and error based method appears never ending. There was always a need to develop a faster, economical method to arrive at the acceptable materials. A machine learning based model has always been immensely useful for such research. Such a model not only advances the search for efficient alloys, but also helps in gaining insights on the underlying chemical process crucial for efficiency.

Several studies have been published that show the effectiveness of a machine learning model in predicting the hydrogen storage capabilities of the materials. In a study by Whitman et.al. titled “Extracting an empirical intermetallic hydride design principle from limited data via interpretable machine learning” published in The Journal of Physical Chemistry Letters 11 (1) (2019) 40-47 present an ML model to predict hydriding characteristics of metal hydrides. Their results reveal the significant reliance of the metal hydride equilibrium H₂ pressure on a volume-based descriptor. Hattrick-Simpers et. al. in “A simple constrained machine learning model for predicting high-pressure-hydrogen-compressor materials”, Mol. Syst. Des. Eng., 2018, 3, 509-517 reported ML model to estimate enthalpy of hydrogenation in metal hydride materials with an mean absolute error (MAE) of 0.09 cV.

US2021/0293381 discloses a method and system of identification of materials for hydrogen storage, wherein a machine learning technique is employed to predict the hydrogen storage capacity of materials, using only the compositional information of the compound. For this, a random forest model is employed which could predict the gravimetric hydrogen storage capacities of intermetallic compounds. The method and system is also configured to predict the thermodynamic stability of the intermetallic compound.

The aforesaid studies focus on predicting the solid-state hydrogen storage capacity using elemental properties of intermetallic compounds. However, absorption temperature influences the capacity to hold hydrogen. Therefore, the inclusion of absorption temperature as a feature for hydrogen storage properties is equally crucial. Also, consideration of thermodynamic properties, such as enthalpy of hydride formation and equilibrium plateau pressure (which is also a function of temperature) along with hydrogen storage capacity, is essential in identifying commercially viable materials for hydrogen storage, as they determine operating temperature/pressure range of hydrogen ab/desorption.

The predicted hydrogen storage capacity changes with absorption temperature. Therefore, it must be predicted as a function of temperature to identify potential storage materials at required temperature which is missing in the above mentioned works.

While there are various systems and methods available for facilitating hydrogen storage computation, there is still a scope for providing an improved solution for identifying hydrogen storage properties of metal alloys.

### Objectives of the Invention

An objective of the present invention to provide a method and system for identifying materials for solid-state hydrogen storage in multi-component metal alloys.

Another objective of the present invention is to provide a method and system to predict solid-state hydrogen storage capacities at different temperatures of multi-component metal alloys with high predictability and ease of operation.

Another objective of the present invention is to provide a method and system to predict enthalpy of hydride formation of multi-component metal alloys with high predictability and ease of interpretation.

Another objective of the present invention is to provide a method and system to predict equilibrium plateau pressure as a function of temperature of multi-component metal alloys with high predictability and ease of interpretation.

### SUMMARY OF THE INVENTION

The present invention relates to the field of hydrogen storage. More particularly, the invention provides a method (100) and system (200) for identification of materials for solid-state hydrogen storage in multi-component metal alloys. Further, the system (200) can predict H₂ storage capacity and equilibrium plateau pressure at different temperatures along with enthalpy of hydride formation of multi-component metal alloys with high predictability and ease of interpretation.

In one aspect of the invention discloses a system (200) for identifying hydrogen storage properties of metal alloys comprising:
(a) an input unit (202) configured for a user to communicate with the system (200);
(b) a database (204) comprising a set of elements; and
(c) a control unit (206)
wherein the system comprising at least one input unit (202), database (204) and one control unit (206) configured to:
access, by the processor (302) of a control unit (206), two or more elements from a database (204), at the input unit (202);
generate, by the processor (302) one or more compositions of AB, AB2, A2B, AB5, solid solution, intermetallics, and High-entropy alloy (HEA) by varying the fractions of the two or more elements in plurality of alloys;
generate, by the processor (302), one or more feature sets representing Metal-Metal and Metal-Hydrogen interactions in each alloy among the plurality of alloys, compositional properties of each alloy, fundamental properties of each alloy, and an absorption temperature of the each alloy;
predict, by the processor (302), a hydrogen weight capacity and an equilibrium plateau pressure at different temperatures and enthalpy of hydrogenation of the one or more alloys based on the one or more feature sets;
identify, by the processor (302), a suitable alloy from the plurality of alloys for hydrogen storage applications based on the hydrogen weight capacity and the equilibrium plateau pressure at different temperatures and enthalpy of hydrogenation; and
display, by the processor (302), the suitable alloy identified from the plurality of alloys, at a user interface of the input unit (202).

In other aspect of the invention, the database (204) comprises 38 elements.

In another aspect of the invention, the control unit (206) comprising a processor (302) coupled with a memory (304), wherein the memory (304) stores one or more instructions executable by the processor (302).

In another aspect of the present invention discloses a method (100) for identifying hydrogen storage properties of metal alloys by system (200) of claim 1 wherein the steps comprises:
(i) accessing the two or more elements comprising accessing a set of elements stored in the database (204),
(ii) generating multi-component metal alloys and a hydrogen storage property of metal alloys from the database (204),
(iii) generating the one or more feature sets comprises a selection of the metal-metal and metal-hydrogen interactions from a group comprising metal-metal dimer bond energy, metal-metal dimer bond length, metal-hydrogen dimer bond energy and metal-hydrogen dimer bond length,
(iv) predicting the hydrogen weight capacity and an equilibrium plateau pressure at different temperatures and enthalpy of hydrogenation of the one or more alloys based on the one or more feature sets;
(v) identifying a suitable alloy from the plurality of alloys for hydrogen storage using an analysis technique,
(vi) displaying, the suitable alloy of step (v) identified from the plurality of alloys, at a user interface of the input unit (202).

In one of the aspect of the present invention the elements are selected from a group comprising Li, Mg, Ca, Al, Si, Ga, Sn, In, Pb, Sc, Ti, V, Cr, Mn, Fe, Co, Ni, Cu, Zn, Y, Zr, Nb, Mo, Rh, Pd, Ag, Hf, Pt, La, Ce Pr, Nd, Sm, Gd, Tb, Dy, Ho and Er.

In another aspect of the present invention, the multi-component metal alloys are selected from different class of alloys AB, AB2, A2B, AB5, solid solution and intermetallics and High-entropy alloys.

In yet another aspect of the present invention, the feature sets comprises a selection of the fundamental properties of the each alloy from a group comprising First Ionization Energy (FIE), Electron Affinity (EA), Atomic Density (AD), Atomic Weight (AW), Boiling Point (BP), Heat of Fusion (HD), Specific Heat (SH), Bulk Modulus (BM), Atomic Molar Volume (AMV), and Thermal Conductivity (TC).

In yet another aspect of the present invention, feature sets comprises a selection of resultant properties of the each alloy are selected from Lattice distortion, entropy of mixing, valence electron concentration and electronegativity difference.

In yet another aspect of the present invention, the analysis technique is selected from a group comprising Linear Regression, Rigid Regression, Kernel Ridge Regression, LASSO Gaussian Process Regression, Extra Tree Regression, Random Forest and Gradient Boosting Regression.

### BRIEF DESCRIPTION OF DRAWINGS

FIG. 1 illustrates a flow diagram depicting a proposed method for facilitating identification of hydrogen storage properties of metal alloys, in accordance with an embodiment of the present disclosure.

FIG. 2 illustrates an exemplary network architecture of the proposed system for facilitating identification of hydrogen storage properties of metal alloys, to illustrate its overall working, in accordance with an embodiment of the present disclosure.

FIG. 3 illustrates exemplary functional units of a control unit associated with the proposed system, in accordance with an exemplary embodiment of the present disclosure.

FIG. 4 illustrates a comparison chart between predicted weight capacities by means of the present system & method and experimentally obtained weight capacities.

FIG. 5 illustrates a chart listing feature importance identified by ETR model for prediction of hydrogen weight percentage.

FIG. 6 illustrates a flowchart demonstrating one or more components of the control unit of the proposed system, accordance with an exemplary embodiment of the present disclosure.

FIG. 7 illustrates a comparison chart between predicted enthalpy of hydride formation by means of the present system & method, and experimentally obtained enthalpy of hydride formation. The error for the final model cross validated over 100 trials was 5.76 KJ/molH2.

FIG. 8 illustrates a chart listing feature importance identified by ETR model for prediction of enthalpy of hydride formation.

FIG. 9 illustrates a comparison chart between predicted equilibrium plateau pressure by means of the present system & method, and experimentally obtained equilibrium plateau pressure. The error for the final model cross validated over 100 trials was 0.58.

FIG. 10 illustrates a chart listing feature importance identified by ETR model for prediction of equilibrium plateau pressure.

FIG. 11 illustrates an exemplary computer system in which or with which embodiments of the present invention can be utilized, in accordance with embodiments of the present disclosure.

### DETAILED DESCRIPTION OF THE INVENTION

In a general embodiment, the present invention provides a method and system for identification of materials for solid-state hydrogen storage in multi-component metal alloys. Further, the system can predict H₂ storage capacity and equilibrium plateau pressure at different temperatures along with enthalpy of hydride formation of multi-component metal alloys with high predictability and ease of interpretation.

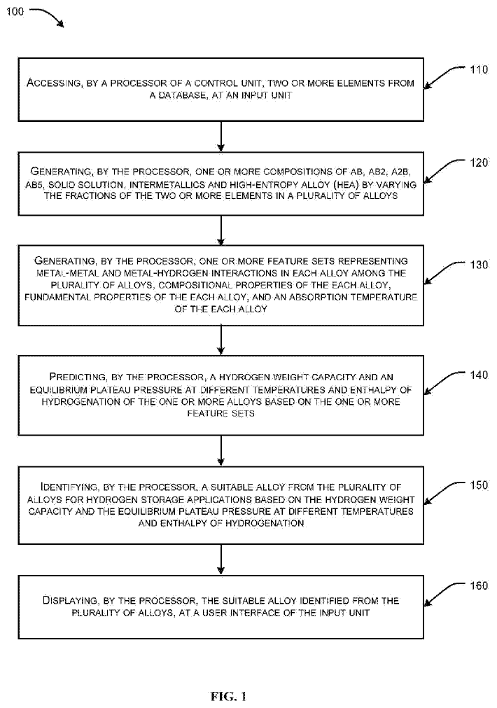

In an embodiment of the present disclosure, referring FIG. 1, a method 100 for identifying hydrogen storage properties of metal alloys comprises the steps of:
At step 110, accessing, by a processor 302 of a control unit 206, two or more elements from a database 204, at an input unit 202;
At step 120, generating, by the processor 302, one or more compositions of AB, AB2, A2B, AB5, solid solution, intermetallics, and High-entropy alloy (HEA) by varying the fractions of the two or more elements in a plurality of alloys;
At step 130, generating, by the processor 302, one or more feature sets representing Metal-Metal and Metal-Hydrogen interactions in each alloy among the plurality of alloys, compositional properties of the each alloy, fundamental properties of the each alloy, and an absorption temperature of the each alloy;
At step 140, predicting 140, by the processor 302, a hydrogen weight capacity and an equilibrium plateau pressure at different temperatures and enthalpy of hydrogenation of the one or more alloys based on the one or more feature sets;
At step 150, identifying, by the processor 302, a suitable alloy from the plurality of alloys for hydrogen storage applications based on the hydrogen weight capacity and the equilibrium plateau pressure at different temperatures and enthalpy of hydrogenation; and
At step 160, displaying, by the processor 302, the suitable alloy identified from the plurality of alloys, at a user interface of the input unit 202.

In an aspect, the one or more compositions comprise a binary, a ternary, and/or a quaternary composition and the database comprises a set of elements comprising 38 elements.

In another embodiment of the present invention, the multi-component metal alloys are selected from a different class of alloys AB, AB2, A2B, AB5, solid solution and intermetallics (binary, ternary, quaternary compositions) and High-entropy alloys (HEA).

In another embodiment of the present invention, the compositions are accessed from a database that stores and provides inputs related to composition and their solid-state hydrogen storage properties. The said database may also be prepared specifically for the purpose of the present invention, combining one or more preexisting databases as well as gathering data from the available literature.

In another embodiment of the present invention, the set of elements include, but are not limited to, Li, Mg, Ca, Al, Si, Ga, Sn, In, Pb, Sc, Ti, V, Cr, Mn, Fe, Co, Ni, Cu, Zn, Y, Zr, Nb, Mo, Rh, Pd, Ag, Hf, Pt, La, Ce Pr. Nd, Sm, Gd, Tb, Dy, Ho, Er.

In another embodiment of the present invention, the interaction based properties of the alloys includes metal-metal interaction and metal-hydrogen interaction. More specifically, metal-metal dimer bond energy, metal-metal dimer bond length, metal-hydrogen dimer bond energy and metal-hydrogen dimer bond length are important properties to be determined. These interactions inside the alloy structure are crucial in understanding the hydrogenation process in an alloy, and therefore are essential factors influencing the material's solid-state hydrogen storage properties.

In another embodiment of the present invention, the fundamental properties of the alloys include First Ionization Energy (FIE), Electron Affinity (EA), Atomic Density (AD), Atomic Weight (AW), Boiling Point (BP), Heat of Fusion (HD), Specific Heat (SH), Bulk Modulus (BM), Atomic Molar Volume (AMV), and Thermal Conductivity (TC). These fundamental elemental properties as features offer good performance of ML models for mapping desired hydrogen storage properties.

In another embodiment of the present invention, the resultant properties of the alloys include Lattice distortion, entropy of mixing, valence electron concentration and electronegativity difference. These features are closely associated with the compositions' structural phase and therefore add more structure-relevant information to the model's learning.

In another embodiment of the present invention, absorption temperature is added as a feature. The hydrogen weight capacity and equilibrium plateau pressure varies substantially with temperature and hence temperature is one of the most important features for predicting hydrogen weight capacity and equilibrium plateau pressure for a given composition.

In another embodiment of the present invention, for determining the suitable materials, the Extra Tree Regression technique is employed.

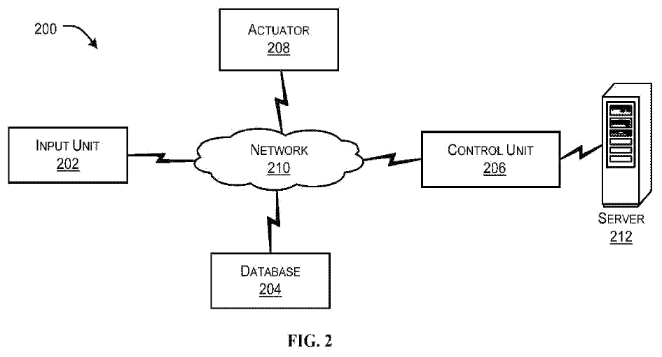

In an embodiment, referring FIG. 2, a system 200 for identifying hydrogen storage properties of metal alloys comprises an input unit 202, a database 204, and a control unit 206. The input unit 202 can be configured for a user to communicate with the system 200. The database 204 can comprise a set of elements. The control unit 206 can be in communication with the input unit 202 and the database 204, the control unit 206 comprising a processor 302 coupled with a memory 304, wherein the memory 304 stores one or more instructions executable by the processor 302 to:
access, by a processor 302 of a control unit 206, two or more elements from the database 204, at the input unit 202;
generate, by the processor 302, one or more compositions of AB, AB2, A2B, AB5, solid solution, intermetallics, and High-entropy alloy (HEA) by varying the fractions of the two or more elements in plurality of alloys;
generate, by the processor 302, one or more feature sets representing Metal-Metal and Metal-Hydrogen interactions in each alloy among the plurality of alloys, compositional properties of the each alloy, fundamental properties of the each alloy, and an absorption temperature of the each alloy;
predict, by the processor 302, a hydrogen weight capacity and an equilibrium plateau pressure at different temperatures and enthalpy of hydrogenation of the one or more alloys based on the one or more feature sets;
identify, by the processor 302, a suitable alloy from the plurality of alloys for hydrogen storage applications based on the hydrogen weight capacity and the equilibrium plateau pressure at different temperatures and enthalpy of hydrogenation; and
display, by the processor 302, the suitable alloy identified from the plurality of alloys, at a user interface of the input unit 202.

In an aspect, the one or more compositions comprise a binary, a ternary, and/or a quaternary composition and the database comprises a set of elements comprising 38 elements.

In another embodiment, the control unit 206 can be further configured to display, via a user interface of the input unit 202, the suitable alloy identified from the plurality of alloys.

In an embodiment, the user interface of the input unit 202 can be configured as a human-machine interface or a machine-machine interface.

In an embodiment, the system 200 can also include an actuator 208, which can be coupled in between the control unit 206, the input unit 202, and the database 204. In an exemplary embodiment, a first signal may be transmitted by the control unit 206, and may then be received by the actuator 208, wherein based on the first signal received; the actuator 108 can enable the processor 302 to execute the one or more instructions stored in the memory 304. Further, the actuator 108 can also enable de-actuation of the input unit 202 via the processor 302 and/or the processor 302, as communicated or commanded by the control unit 206 on reception of a user input or based on the one or more instructions stored in the memory 304.

In an embodiment, the control unit 206 can be in communication with the input unit 202, the database 204, and the actuator 208, through a network 210. Further, the network 210 can be a wireless network, a wired network or a combination thereof that can be implemented as one of the different types of networks, such as Intranet, Local Area Network (LAN), Wide Area Network (WAN), Internet, and the like. Furthermore, the network 210 can either be a dedicated network or a shared network. The shared network can represent an association of different types of networks that can use variety of protocols, for example, Hypertext Transfer Protocol (HTTP), Transmission Control Protocol/Internet Protocol (TCP/IP), Wireless Application Protocol (WAP), and the like.

In an embodiment, the system 200 can be implemented using any or a combination of hardware components and software components such as a cloud, a server 212, a computing system, a computing device, a network device and the like. Further, the control unit 206 can interact with the input unit 202, the database 204, and the actuator 208, through a website or an application that can reside in the proposed system 200. In an implementation, the proposed system 200 can be accessed by website or application that can be configured with any operating system, including but not limited to, Android™, iOS™, and the like.

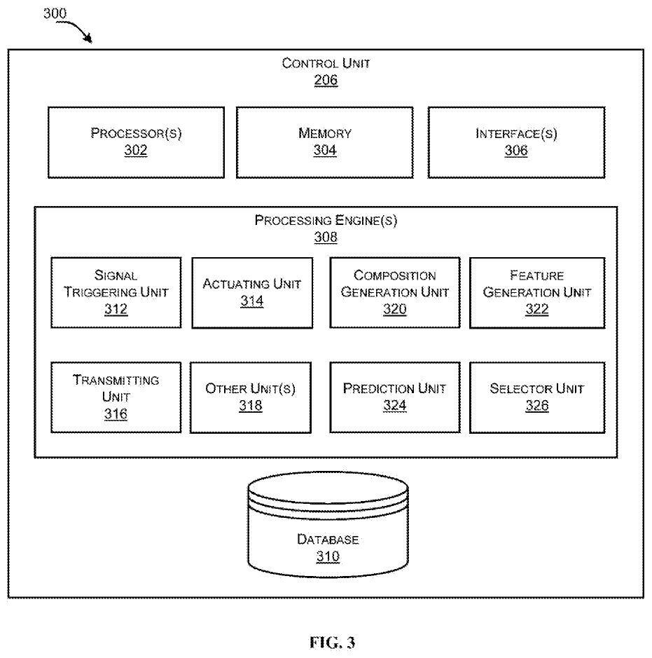

Referring to FIG. 3, block diagram 300 represents exemplary functional units of the control unit 206. The control unit 206 can include one or more processor(s) 302. The one or more processor(s) 302 can be implemented as one or more microprocessors, microcomputers, microcontrollers, digital signal processors, central processing units, logic circuitries, and/or any devices that manipulate data based on operational instructions. Among other capabilities, the one or more processor(s) 302 are configured to fetch and execute computer-readable instructions stored in a memory 304 of the control unit 206. The memory 304 can store one or more computer-readable instructions or routines, which may be fetched and executed to create or share the data units over a network service. The memory 304 can include any non-transitory storage device including, for example, volatile memory such as RAM, or non-volatile memory such as EPROM, flash memory, and the like.

In an embodiment, the control unit 206 can also include an interface(s) 306. The interface(s) 306 may include a variety of interfaces, for example, interfaces for data input and output devices, referred to as I/O devices, storage devices, and the like. The interface(s) 306 may facilitate communication of the monitoring device with various devices coupled to the control unit 206. The interface(s) 306 may also provide a communication pathway for one or more components of the control unit 206. Examples of such components include, but are not limited to, processing engine(s) 308 and database 310.

In an embodiment, the processing engine(s) 308 can be implemented as a combination of hardware and programming (for example, programmable instructions) to implement one or more functionalities of the processing engine(s) 308. In examples described herein, such combinations of hardware and programming may be implemented in several different ways. For example, the programming for the processing engine(s) 308 may be processor executable instructions stored on a non-transitory machine-readable storage medium and the hardware for the processing engine(s) 308 may include a processing resource (for example, one or more processors), to execute such instructions.

In the present examples, the machine-readable storage medium may store instructions that, when executed by the processing resource, implement the processing engine(s) 308. In such examples, the control unit 206 can include the machine-readable storage medium storing the instructions and the processing resource to execute the instructions, or the machine-readable storage medium may be separate but accessible to the system 200 and the processing resource. In other examples, the processing engine(s) 308 may be implemented by electronic circuitry. The database 310 can include data that is either stored or generated as a result of functionalities implemented by any of the components of the processing engine(s) 308. In an embodiment, the processing engine(s) 208 can include a signal triggering unit 312, an actuating unit 314, a transmitting unit 316, and other units(s) 318. The other unit(s) 318 can implement functionalities that supplement applications/functions performed by the control unit 206.

In an embodiment, the processing engine(s) 208 can include a composition generation unit 320 for generating the one or more compositions, a feature generation unit 322 for generating the one or more feature sets, a prediction unit 324 for predicting the hydrogen weight capacity and the equilibrium plateau pressure at different temperatures and enthalpy of hydrogenation of the one or more alloys, and a selector unit 326 for identifying the suitable alloy.

According to an embodiment, the signal triggering unit 312 can trigger a first signal for execution of the one or more instructions stored in the memory 304, upon receiving a request for the execution of the one or more instructions by the user or the one or more processor(s) 302.

According to an embodiment, the actuating unit 314 can enable selective execution of the one or more instructions stored in the memory 304, based on the received first signal, for facilitating easy and smooth execution of the one or more instructions by the one or more processor(s) 302.

In another embodiment, the actuating unit 314 can also enable de-actuation of the input unit 202 via the processor 302 and/or the processor 302, as communicated or commanded by the control unit 206 on reception of a user input or based on the one or more instructions stored in the memory 304.

According to an embodiment, the transmitting unit 316 can facilitate transmission of signals and/or requests of the user or the processor 302 for actuating the input unit 202.

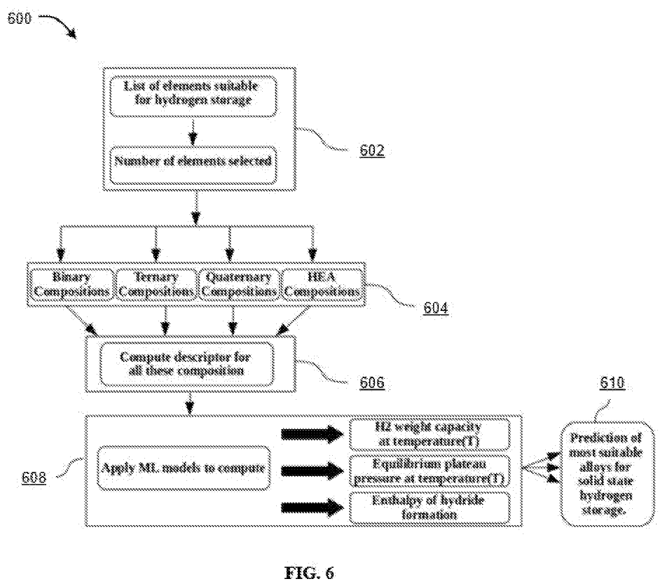

Referring FIG. 6, in an embodiment, the system 600, identical with the system 100, comprises an input unit 602 (identical with 102), the composition generation unit 604 (identical with 320) for generating the one or more compositions, a feature generation unit 606 (identical with 322) for generating the one or more feature sets, a prediction unit 608 (identical with 324) for predicting the hydrogen weight capacity and the equilibrium plateau pressure at different temperatures and enthalpy of hydrogenation of the one or more alloys, and a selector unit 610 (identical with 326) for identifying the suitable alloy, as a separate units other than the control unit 206.

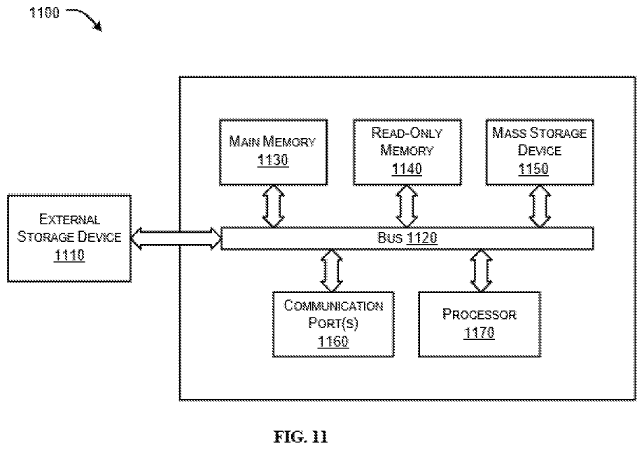

Referring to FIG. 11, block diagram 1100 represents a computer system that includes an external storage device 1110, a bus 1120, a main memory 1130, a read only memory 1140, a mass storage device 1150, communication port 1160, and a processor 1170. A person skilled in the art will appreciate that computer system may include more than one processor and communication ports. Examples of processor 670 include but are not limited to, an Intel® Itanium® or Itanium 2 processor(s), or AMD® Opteron® or Athlon MP® processor(s), Motorola® lines of processors, FortiSOC™ system on a chip processors or other future processors. Processor 1170 may include various modules associated with embodiments of the present invention. Communication port 1160 can be any of an RS-232 port for use with a modem based dialup connection, a 10/100 Ethernet port, a Gigabit or 10 Gigabit port using copper or fiber, a serial port, a parallel port, or other existing or future ports. Communication port 660 may be chosen depending on a network, such a Local Area Network (LAN), Wide Area Network (WAN), or any network to which computer system connects.

In an embodiment, the memory 1130 can be Random Access Memory (RAM), or any other dynamic storage device commonly known in the art. Read only memory 1140 can be any static storage device(s) e.g., but not limited to, a Programmable Read Only Memory (PROM) chips for storing static information e.g., start-up or BIOS instructions for processor 1170. Mass storage 1160 may be any current or future mass storage solution, which can be used to store information and/or instructions. Exemplary mass storage solutions include, but are not limited to, Parallel Advanced Technology Attachment (PATA) or Serial Advanced Technology Attachment (SATA) hard disk drives or solid-state drives (internal or external, e.g., having Universal Serial Bus (USB) and/or Firewire interfaces), e.g. those available from Seagate (e.g., the Seagate Barracuda 7102 family) or Hitachi (e.g., the Hitachi Deskstar 7K1000), one or more optical discs, Redundant Array of Independent Disks (RAID) storage, e.g. an array of disks (e.g., SATA arrays), available from various vendors including Dot Hill Systems Corp., LaCie, Nexsan Technologies, Inc. and Enhance Technology, Inc. In an embodiment, the bus 1120 communicatively couples processor(s) 1170 with the other memory, storage, and communication blocks. Bus 1120 can be, e.g. a Peripheral Component Interconnect (PCI)/PCI Extended (PCI-X) bus, Small Computer System Interface (SCSI), USB, or the like, for connecting expansion cards, drives, and other subsystems as well as other buses, such a front side bus (FSB), which connects processor 1170 to software system.

In another embodiment, operator and administrative interfaces, e.g. a display, keyboard, and a cursor control device, may also be coupled to bus 1120 to support direct operator interaction with computer system. Other operator and administrative interfaces can be provided through network connections connected through communication port 1160. External storage device 1110 can be any kind of external hard-drives, floppy drives, IOMEGA® Zip Drives, Compact Disc-Read Only Memory (CD-ROM), Compact Disc-Re-Writable (CD-RW), Digital Video Disk-Read Only Memory (DVD-ROM). Components described above are meant only to exemplify various possibilities. In no way should the aforementioned exemplary computer system limit the scope of the present disclosure.

In yet another embodiment, the present invention provides a method and system for identifying materials for hydrogen storage based on crucial hydrogen storage properties of multi-component metal alloy. Further, the present invention discloses a method and system to predict the temperature dependent hydrogen storage weight capacity using periodic table properties and parameters that can be easily compounded with minimal computation. The present invention thus discloses a predictive method and system that governs metal-hydrogen interaction, to understand the hydrogen storage properties of any alloy.

In another embodiment, the present invention introduces new features relevant for prediction of hydrogen storage properties. Thus, providing a method and system that can advance the search for efficient alloys and also help in gaining insights on the underlying chemical process is crucial for efficiency.

In another embodiment of the present invention, the advantage of the system can predict H₂ storage capacity and equilibrium plateau pressure at different temperatures, along with enthalpy of hydride formation with high predictability and ease of interpretation.

### Examples

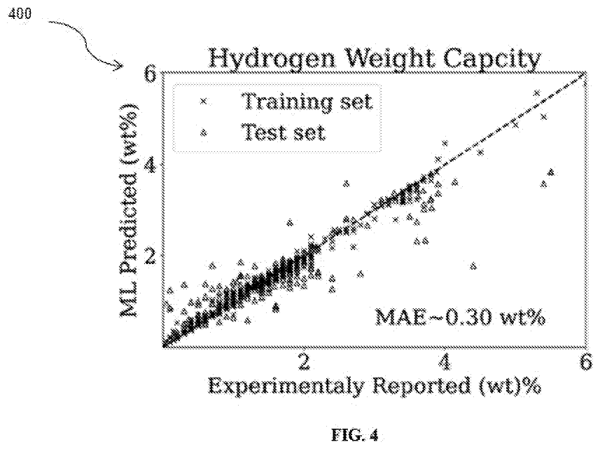

FIG. 4 illustrates a comparison chart between predicted weight capacities by means of the present system & method, and experimentally obtained weight capacities. The error for the final model cross validated over 100 trials was 0.30 wt %.

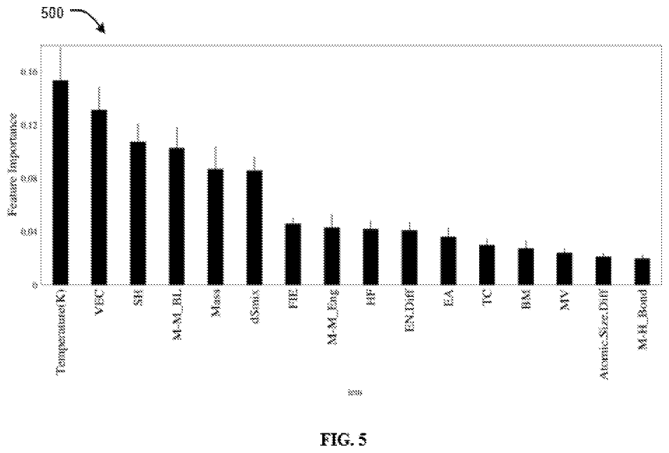

FIG. 5 illustrates a chart listing feature importance identified by ETR model for prediction of hydrogen weight capacity. The temperature feature has the highest importance for prediction of hydrogen weight capacity.

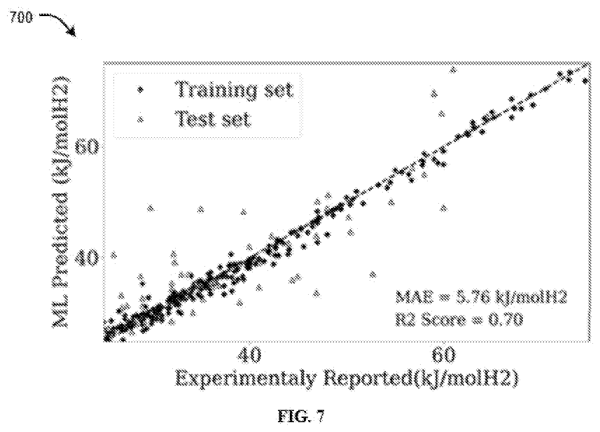

FIG. 7 illustrates a comparison chart between predicted enthalpy of hydride formation by means of the present system & method, and experimentally obtained enthalpy of hydride formation. The error for the final model cross validated over 100 trials was 5.76 KJ/molH2.

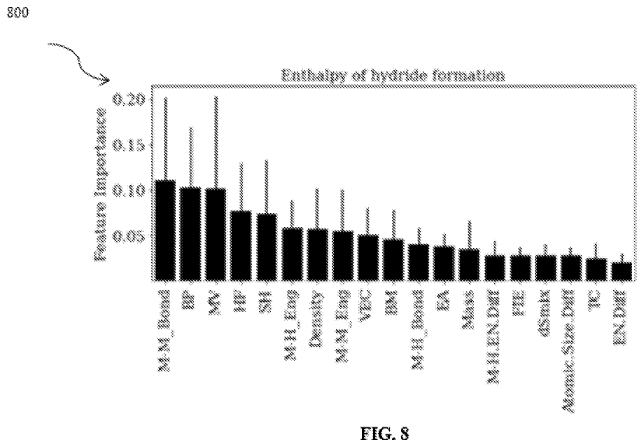

FIG. 8 illustrates a chart listing feature importance identified by ETR model for prediction of enthalpy of hydride formation.

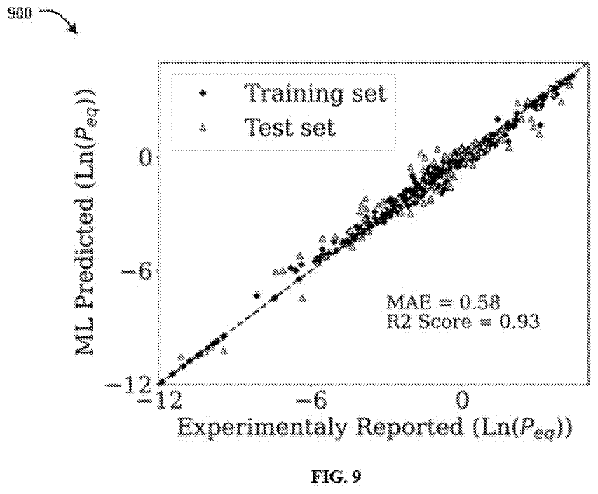

FIG. 9 illustrates a comparison chart between predicted equilibrium plateau pressure by means of the present system & method, and experimentally obtained equilibrium plateau pressure. The error for the final model cross validated over 100 trials was 0.58.

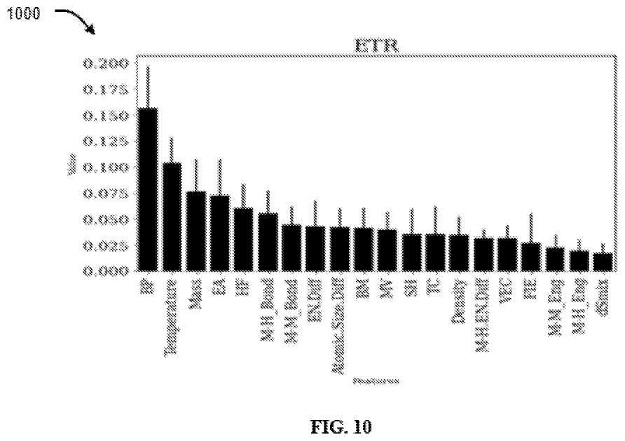

FIG. 10 illustrates a chart listing feature importance identified by ETR model for prediction of equilibrium plateau pressure.

The examples demonstrating improvement in hydrogen weight capacity with addition of temperature are described in Table 1 and Table 2.

Table 1 illustrates error bars for train, test, and validation set for temperature in/dependent model for hydrogen weight capacity. Temperature as a feature has significantly improved predictability. For H2 wt %, the inclusion of temperature as a feature has significantly improved the model's performance compared to the temperature-independent model, as tabulated in Table 1. A considerable rise in R2 score for the validation set (unseen data) is observed when temperature is included as one of the features.

TABLE 1

| | MAE | R2 | MAE | R2 Score |
|---|---|---|---|---|
| Model | test | Score | Validation | Validation |
| Temperature | 0.35 | 0.73 | 0.53 | 0.64 |
| Independent (H2 wt %) | | | | |
| Temperature | 0.31 | 0.81 | 0.44 | 0.81 |
| Dependent (H2 wt %) | | | | |

Table 2 illustrates composition, temperature, experimentally reported hydrogen weight capacity at that temperature, ML (temperature independent model) predicted, and ML (Temperature dependent model) predicted hydrogen weight capacity to bring out the effect of temperature on the model's prediction. In the absence of temperature as one of the features, the model predicts the same value at different temperatures whereas the temperature dependent model picks up the variation due to temperature quite well. The temperature-independent model could not capture the variation observed in H2 wt % with temperature. In contrast, the temperature dependent model has predicted H2 wt % as a function of temperature for a given composition which is at par with the experimentally reported results.

TABLE 2

| | | Experi- | T-independent | T-dependent |
|---|---|---|---|---|
| | | mentally | model | model |
| Composition | T | Reported | predictions | predictions |
| Formula | (K) | (wt %) | (wt %) | (wt %) |
| Mg-30 wt % LaNi5 | 470 | 3.8 | 5.00 | 3.78 |
| Mg-30 wt % LaNi5 | 623 | 4.66 | 5.00 | 5.14 |
| Mg88Y12 | 373 | 2.63 | 5.08 | 2.72 |
| Mg88Y12 | 423 | 3.94 | 5.08 | 3.32 |
| Mg88Y12 | 653 | 6 | 5.08 | 5.14 |

### ADVANTAGES OF THE PRESENT INVENTION

1. The present invention provides an easy and efficient prediction of crucial hydrogen storage properties for any alloy composition.
2. The present invention provides a system which can predict hydrogen weight capacity at different temperatures for any alloy composition
3. The present invention provides a system which can predict equilibrium plateau pressure at different temperatures for any alloy composition.
4. The present invention provides a system which can predict enthalpy of hydrogenation for any alloy composition.
5. The present invention provides an easy and efficient prediction of the suitable materials for solid state hydrogen storage.

1. A system (200) for identifying hydrogen storage properties of metal alloys comprising:
(a) an input unit (202) configured for a user to communicate with the system (200);
(b) a database (204) comprising a set of elements; and
(c) a control unit (206)
wherein the system comprising at least one input unit (202), database (204) and one control unit (206) is configured to:
access, by the processor (302) of a control unit (206), two or more elements from a database (204), at the input unit (202);
generate, by the processor (302) one or more compositions of AB, AB2, A2B, AB5, solid solution, intermetallics, and High-entropy alloy (HEA) by varying the fractions of the two or more elements in plurality of alloys;
generate, by the processor (302), one or more feature sets representing Metal-Metal and Metal-Hydrogen interactions in each alloy among the plurality of alloys, compositional properties of each alloy, fundamental properties of each alloy, and an absorption temperature of the each alloy;
predict, by the processor (302), a hydrogen weight capacity and an equilibrium plateau pressure at different temperatures and enthalpy of hydrogenation of the one or more alloys based on the one or more feature sets;
identify, by the processor (302), a suitable alloy from the plurality of alloys for hydrogen storage applications based on the hydrogen weight capacity and the equilibrium plateau pressure at different temperatures and enthalpy of hydrogenation; and
display, by the processor (302), the suitable alloy identified from the plurality of alloys, at a user interface of the input unit (202).

2. The system as claimed in claim 1, wherein the database (204) comprises 38 elements.

3. The system as claimed in claim 1, wherein the control unit (206) comprising a processor (302) coupled with a memory (304), wherein the memory (304) stores one or more instructions executable by the processor (302).

4. A method (100) for identifying hydrogen storage properties of metal alloys by system (200) of claim 1 comprising:
(i) accessing the two or more elements comprising a set of elements stored in the database (204),
(ii) generating multi-component metal alloys and a hydrogen storage property of metal alloys from the database (204),
(iii) generating the one or more feature sets comprises a Metal-Metal and Metal-Hydrogen interactions in each alloys, compositional properties of each alloys, fundamental properties of each alloys, and an absorption temperature of each alloys,
(iv) predicting the hydrogen weight capacity and an equilibrium plateau pressure at different temperatures and enthalpy of hydrogenation of one or more alloys based on the one or more feature sets;
(v) identifying a suitable alloy from the plurality of alloys for hydrogen storage based on said predicted hydrogen weight capacity and equilibrium plateau pressure, using an analysis technique,
(vi) displaying, the suitable alloy of step (v) identified from the plurality of alloys, at a user interface of the input unit (202).

5. The method as claimed in claim 4, wherein the elements are selected from a group comprising Li, Mg, Ca, Al, Si, Ga, Sn, In, Pb, Sc, Ti, V, Cr, Mn, Fe, Co, Ni, Cu, Zn, Y, Zr, Nb, Mo, Rh, Pd, Ag, Hf, Pt, La, Ce Pr, Nd, Sm, Gd, Tb, Dy, Ho and Er.

6. The method as claimed in claim 4, wherein the multi-component metal alloys are selected from different class of alloys AB, AB2, A2B, AB5, solid solution and intermetallics and High Entropy Alloys.

7. The method as claimed in claim 4, wherein the one or more feature sets essentially comprises a selection of the metal-metal and metal-hydrogen interactions from a group comprising metal-metal dimer bond energy, metal-metal dimer bond length, metal-hydrogen dimer bond energy and metal-hydrogen dimer bond length.

8. The method as claimed in claim 4, wherein the feature sets comprises a selection of the fundamental properties of the each alloy from a group comprising First Ionization Energy (FIE), Electron Affinity (EA), Atomic Density (AD), Atomic Weight (AW), Boiling Point (BP), Heat of Fusion (HD), Specific Heat (SH), Bulk Modulus (BM), Atomic Molar Volume (AMV), and Thermal Conductivity (TC).

9. The method as claimed in claim 4, wherein the feature sets comprise a selection of resultant properties of the each alloy that are selected from Lattice distortion, entropy of mixing, valence electron concentration and electro-negativity difference.

10. The method as claimed in claim 4, wherein the analysis technique is selected from a group comprising Linear Regression, Rigid Regression, Kernel Ridge Regression, LASSO Gaussian Process Regression, Extra Tree Regression, Random Forest and Gradient Boosting Regression.
